(19) United States
(12) Patent Application Publication
(10) Pub. No.: US 2016/0166505 A1
(43) Pub. Date: Jun. 16, 2016

(54) Methods And Compositions For Treatment Of Respiratory Tract Infections

(71) Applicant: CMPD LICENSING, LLC, Conroe, TX (US)

(72) Inventor: Jay Richard Ray, II, Conroe, TX (US)

(21) Appl. No.: 14/566,313

(22) Filed: Dec. 10, 2014

(51) Int. Cl. A61K 9/00; A61K 9/48; A61K 31/198; A61K 31/7036

(52) U.S. Cl. CPC A61K 9/0078; A61K 31/7036; A61K 9/48; A61K 31/198

(57) ABSTRACT

A method of treating a patient for an upper or lower respiratory tract infection includes mixing a dry powder pharmaceutical composition including tobramycin with a base liquid to produce a nebulizer solution dosage form. The method may further include encapsulating the powder in a capsule and dispensing the capsule for the subsequent mixing of the powder with the base prior to administration to the patient via intranasal nebulization.

### TECHNICAL FIELD

The present application relates to compounded therapies. In particular, the present application relates to compositions and methods for compounded therapy of respiratory tract infections.

### BACKGROUND

Respiratory tract infections are among the most common ailments suitable for medicinal treatment. Juggling the need for therapeutic efficacy and patient compliance with respect to such treatments, however, is an ongoing problem with very few if any instances of one size fits all solutions. Pharmacokinetics for instance may complicate optimal drug delivery and efficacy. For example, while various medicinal compositions are known for oral administration in the form of pills or solutions for ingestion and subsequent absorption in the gut, such administration may be inadequate for other compositions that are poorly absorbed. In some instances, medicinal compositions may be injected into veins or muscle. Many patients, however, are uncomfortable with self-administering or receiving such injections. Additionally, such medications often have to be administered in the hospital (often in the intensive-care-unit) or, at minimum, through home-healthcare-services. Such treatment aspects are not only incredibly invasive but also costly. Accordingly, despite existence of potential administration routes, therapy options may remain limited due to patient preference or costs.

What is needed are additional therapy options to treat respiratory tract infections and improve patient outcomes.

### SUMMARY

In one aspect a method of treating a patient for an upper or lower respiratory tract infection comprises preparing a pharmaceutical composition comprising a dry powder formulation comprising tobramycin formulated to be mixed with water for injection or sodium chloride solution to produce a nebulizer solution dosage form. The method may further comprise encapsulating the dry powder formulation in a capsule and dispensing the encapsulated dry powder formulation for subsequent addition of the dry powder formulation to the water for injection or sodium chloride solution to produce the nebulizer solution dosage form for administration to the patient via intranasal nebulization to treat the infection of the upper or lower respiratory tract.

In various embodiments, preparing the pharmaceutical composition comprises combining the tobramycin with a mucolytic agent to form the dry powder formulation. In one embodiment, the mucolytic agent comprises acetylcysteine or a pharmaceutically acceptable salt thereof. Encapsulating the dry powder may comprise encapsulating the acetylcysteine and approximately 100 mg of tobramycin or approximately 150 mg of tobramycin sulfate in the capsule. Encapsulating the dry powder may further comprise encapsulating the tobramycin and approximately 20 mg of acetylcysteine in the capsule. Preparing the pharmaceutical composition may further comprises obtaining at least one of the tobramycin and the acetylcysteine from a bulk source.

In one embodiment, preparing the pharmaceutical composition comprises combining the tobramycin with an excipient base comprising LoxaSperse to form the dry powder formulation. Encapsulating the dry powder may comprise encapsulating the LoxaSperse and approximately 100 mg of tobramycin or approximately 150 mg of tobramycin sulfate in the capsule. Preparing the pharmaceutical composition may further comprise obtaining the tobramycin from a bulk source. The dry powder formulation may further comprise a mucolytic agent. The mucolytic agent may comprise acetylcysteine or a pharmaceutically acceptable salt thereof. Encapsulating the dry powder formulation may comprise encapsulating the LoxaSperse and approximately 100 mg of tobramycin or approximately 150 mg of tobramycin sulfate and approximately 20 mg of acetylcysteine in the capsule. Preparing the pharmaceutical composition may further comprise obtaining at least one of the tobramycin and the acetylcysteine from a bulk source.

In another aspect, a pharmaceutical composition comprises a dry powder formulation encapsulated within a capsule. The dry powder formulation may be formulated to be mixed with water for injection or sodium chloride solution to produce a nebulizer solution dosage form for nebulization and intranasal administration to a patient to treat the infection of the upper or lower respiratory tract. The dry powder formulation may comprise tobramycin and at least one of a mucolytic agent and an excipient base comprising LoxaSperse.

In one embodiment, the pharmaceutical composition comprises tobramycin in an amount of approximately 100 mg or approximately 150 mg of tobramycin sulfate. The mucolytic agent may comprise a pharmaceutically effective amount of acetylcysteine or a pharmaceutically acceptable salt thereof. In one embodiment, the tobramycin may be present in an amount of approximately 100 mg or approximately 150 mg of tobramycin sulfate and the acetylcysteine may be present in an amount of approximately 20 mg. In some embodiments, at least one of the tobramycin and the acetylcysteine is derived from a bulk source.

In another aspect, a method of treating a patient for an upper or lower respiratory tract infection comprises mixing the dry powder formulation with the water for injection or sodium chloride solution to prepare the nebulizer solution dosage form for nebulization and intranasal administration to the patient to treat the infection of the upper or lower respiratory tract.

In one embodiment, the mucolytic agent comprises a pharmaceutically effective amount of acetylcysteine or a pharmaceutically acceptable salt thereof. The method may further comprise removing the dry powder formulation from a capsule encapsulating the dry powder formulation prior to mixing the dry powder formulation with the water for injection or sodium chloride solution.

In yet another aspect, a method of treating an upper or lower respiratory tract infection comprises administering the nebulizer solution dosage form by nebulizing the solution with a small particle size nebulizer to produce nebulized particles of the nebulizer solution dosage form for administration to the patient via intranasal nebulization to treat the infection of the upper or lower respiratory tract. In one embodiment, nebulizing the nebulizer solution dosage form comprises producing a plurality of nebulized particles wherein a majority of the nebulized particles are less than 10 microns in size. In one embodiment, the nebulizer solution dosage form comprises approximately 100 mg or approximately 150 mg of tobramycin sulfate and approximately 20 mg of acetylcysteine.

### DESCRIPTION

The present embodiments may generally relate to pharmaceutical compositions formulated for the treatment of one or more ailments of the respiratory tract, such as infections of the lower or upper respiratory tract. The respiratory tract infection treated may include a condition caused by or associated with a bacterial, viral, or fungal infection, including a Pseudomonas infection or a MRSA infection, influenza or the common cold. Other conditions treated may include cystic fibrosis, bronchiolitis, bronchiectasis, tracheobronchitis, pneumonia (including ventilator-associated pneumonia, pneumonitis, dyspnea, cough, (recurrent) wheezing, asthma, nasal polyopsis, allergic rhinitis, upper respiratory infections (Common cold), pulmonary sarcoidosis, anosmia, olfactory (smell) loss, sinus ostia stenosis, aspergilliosis, pulmonary invasive fungal infections, sinusitis, chronic rhinosinusitis, nosocomial lung infections.

The pharmaceutical composition comprises one or more active agents comprising an anti-infective or antimicrobial agent (e.g., antibiotic, antiviral, antiparacytic, antifungal, etc.) alone or in combination with an additional anti-infective or antimicrobial agent or other active agent such as an anti-inflammatory or mucolytic agent.

The pharmaceutical composition may comprise, for example, a formulation of compounded medications. The composition may be formulated as a dry powder comprising one or more compounded active agents. In one embodiment, the dry powder may be encapsulated to provide an encapsulated dosage form, which, in some embodiments, may comprise a unit dose or unit dose form. The encapsulated dosage form may be suitable for administration to the patient or may be configured for further manipulation to produce an administration dosage form, e.g., by addition of the dry powder to a liquid. For example, in one embodiment, a first dosage form comprising the dry powder is formulated to be added to a base to produce a second administration dosage form suitable to be administered to the patient for topical application or local action at an infection site, such as an infected respiratory surface. In various embodiments, the administration dosage form comprises a mixture, suspension, emulsion, or solution, for example, prepared from the dry powder and a base. In some embodiments, the base may comprise an aqueous solution, which may include a diluent, solvent, or liquid medium.

In various embodiments, the one or more active agents comprise an antibiotic. The antibiotic may include one or more antibiotics selected from amikacin, amoxicillin, gentamicin, kanamycin, neomycin, netilmicin, paromomycin, tobramycin, geldanamycin, herbimycin, carbacephem (loracarbef), ertapenem, doripenem, imipenem, cefadroxil, cefazolin, cefalotin, cephalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole, clarithromycin, clavulanic acid, clindamycin, colistimethate teicoplanin, azithromycin, dirithromycin, erythromycin, troleandomycin, telithromycin, aztreonam, ampicillin, azlocillin, bacampicillin, carbenicillin, cloxacillin, dicloxacillin, flucloxacillin, mezlocillin, meticillin, nafcillin, norfloxacin, oxacillin, penicillin G, penicillin V, piperacillin, pvampicillin, pivmecillinam, ticarcillin, bacitracin, colistin, colimycin, polymyxin B, ciprofloxacin, enoxacin, gatifloxacin, levofloxacin, lomefloxacin, moxifloxacin, ofloxacin, trovafloxacin, grepafloxacin, sparfloxacin, afenide, prontosil, sulfacetamide, metronidazole, sulfamethizole, sulfanilimide, sulfamethoxazole, sulfisoxazole, trimethoprim, trimethoprim-sulfamethoxazole, demeclocycline, doxycycline, oxytetracycline, tetracycline, arsphenamine, chloramphenicol, chlorhexidine, lincomycin, ethambutol, fosfomycin, furazolidone, isoniazid, linezolid, mupirocin, nitrofurantoin, platensimycin, pyrazinamide, quinupristin/dalfopristin, rifampin, thiamphenicol, rifampicin, minocycline, sultamicillin, sulbactam, sulphonamides, mitomycin, spectinomycin, spiramycin, roxithromycin, and meropenem.

In a preferred embodiment, the antibiotic comprises an aminoglycoside. Aminoglycosides generally target gram-negative bacteria via inhibition of protein synthesis. Aminoglycosides are considered to be poorly absorbed when administered orally but are reported to be well absorbed from denuded skin and the peritoneum, pleural cavity, and joints, but their use is discouraged to instill in these body cavities. Due to poor absorption and inability to pass the gastro-intestinal tract, aminoglycosides are typically administered intravenously and intramuscularly.

The pharmaceutical composition disclosed herein may preferably include an aminoglycoside in the form of a Streptomyces derived aminoglycoside such as streptomycin, deoxystreptamine-containing agents kanamycin, tobramycin, gentamicin, or neomycin. In certain preferred embodiments, the aminoglycoside comprises tobramycin. Tobramycin is an aminoglycoside antibiotic that may be derived from Streptomyces tenebrarius and may be used to treat various types of bacterial infections, particularly Gram-negative infections. It is especially effective against species of Pseudomonas.

In a solid or powdered format tobramycin has a white to off-white appearance. Due to its characteristic poor absorption consistent with the aminoglycoside class, systemic use of tobramycin is believed to be limited to intravenous and intramuscular injection while nebulization formulations are also available. Tobramycin for injection (aka Nebcin, tobramycin sulfate) is commercially available in a bulk dry powder including 2.5 g vials for dilution. Additional formulations having low systemic absorption include ophthalmic formulations and nebulized formulations. A 0.3% tobramycin solution is currently marketed under the name “TOBREX®”. An aqueous suspension containing 0.3% tobramycin and 0.1% dexamethasone is currently marketed under the name “TOBRADEX®”. Both of the above-mentioned products are preserved by means of benzalkonium chloride. TOBREX® is a 0.3% tobramycin sterile ophthalmic solution that includes benzalkonium chloride 0.01% as a preservative. TOBRADEX® is a tobramycin and dexamethasone ophthalmic suspension that contains per mL tobramycin 0.3% and the corticosteroid dexamethasone 0.1%, benzalkonium chloride 0.01% as a preservative, and the inactives: tyloxapol, edetate disodium, sodium chloride, hydroxyethyl cellulose, sodium sulfate, sulfuric acid and/or sodium hydroxide (to adjust pH) and purified water. TOBI® is a nebulizer solution of tobramycin in the form of a 300 mg/5 mL tobramycin including sodium chloride, water for injection, and sulfuric acid/sodium hydroxide for pH adjustment.

In various embodiments, the aminoglycoside of the pharmaceutical composition comprises tobramycin. The tobramycin may be provided in a solid or dry powder. Suitable dry powder forms of tobramycin may be derived from commercially available bulk sources. The tobramycin may be compressed in a tablet form or encapsulated alone or in combination with one or more additional active agents or one or more pharmaceutically acceptable excipients. For example, in one embodiment, composition comprises multiple aminoglycosides such as tobramycin and at least one additional aminoglycoside and one or more pharmaceutically acceptable excipients. Other active agents may be added for purposes of alleviating other undesirable conditions associated with an administration or application of the composition such as at a body orifice, respiratory tract surface, or along a delivery pathway. Such agents may include, for example, anesthetics, analgesics, anti-inflammatories, antibacterial agents, antiviral agents and emollients.

In various embodiments, the composition comprises an aminoglycoside and one or more additional active agents comprising a mucolytic agent. Mucolytics are used to dissolve or breakdown mucus in the respiratory tract. Mucolytics generally act on mucus to reduce its viscosity thereby increasing the effectiveness of coughing. Accordingly, in one embodiment, the composition includes tobramycin and one or more mucolytics formulated to treat respiratory conditions such as upper or lower respiratory tract infections, especially those associated with excessive or thickened mucus secretion. Various embodiments of the composition disclosed herein may include one or more mucolytic agents known in the art. In some embodiments, the one or more mucolytic agents may be selected from the group consisting of acetylcysteine, erdosteine, bromheksin, carbocisteine and guiafenesin or pharmaceutically acceptable salts thereof.

In one embodiment, the formulation comprises the aminoglycoside tobramycin and the mucolytic agent acetylcysteine. One or both of tobramycin and acetylcysteine may be derived from bulk sources. One or both of tobramycin and acetylcysteine may be present in the composition as a dry powder. For example, the composition may comprise a dry powder formulation comprising tobramycin and acetylcysteine, which may further include one or more pharmaceutically acceptable excipients. Tobramycin may be included at approximately 100 mg per unit, e.g., encapsulated within a capsule, or approximately 150 mg tobramycin sulfate per unit. In one embodiment, the total daily dose of Tobramycin will be approximately 600 mg. The percent composition of tobramycin in the composition may be greater than that of acetylcysteine. In some embodiments, tobramycin may be present in the dry powder formulation in amounts that are in multiples, e.g., two, three, four, five, or more times that of the amount of acetylcysteine present in the composition.

In some embodiments, the dry powder formulation may comprise additional active agents. For example, the dry powder formulation may comprise an antibiotic, such as the aminoglycoside tobramycin, and one or more additional antibiotics selected from quinolones, mupirocin, vancomycin, tetracycline, chlortetracycline, bacitracin, neomycin, polymyxin, gramicidin, cephalexin, oxytetracycline, chloramphenicol, rifampicin, ciprofloxacin, gentamycin, erythromycin, penicillin, sulfa-based agents, sulfonamides, sulfadiazine, sulfacetamide, sulfamethizole, sulfisoxazole, nitrofurazone, sodium propionate, or other antibiotics or derivatives thereof. The dry powder formulation according to the above may further comprise the mucolytic agent, such as acetylcysteine.

According to various embodiments, the dry powder formulation comprises an antibiotic and an antifungal agent. Preferred antifungal agents include, but are not limited to, nystatin, ciclopirox, amphotericin B, flucytosine, and terbinafine. Preferred antifungal agents may also include one or more azoles selected from clotrimazole, econazole, oxiconazole, ketoconazole, miconazole, fluconazole, itraconazole, amorolfineitraconazole, and voriconazole. According to various embodiments, composition comprises a dry powder formulation including an antibiotic aminoglycoside tobramycin and one or more active agents comprising one or more of the antifungal agents selected from above. In a further embodiment, the dry powder formulation comprises the mucolytic acetylcysteine.

In certain embodiments, the pharmaceutical composition may comprise the one or more active agents alone or the one or more active agents and one or more pharmaceutically acceptable excipients. One or more of such excipients may be formulated to assist in the release, dispersion, solubility, or the delivery of one or more of the active agents at the administration or target site, e.g., skin, lungs, respiratory surfaces, or one or more body orifices. For example, excipients may include synthetic or naturally derived excipients such as one or more of a base, solvent, surfactant, permeation enhancer, emollient, humectant, disintegrate, acid, base, pH modifying agent or buffer, solubility enhancer, or a carrier molecule or complex configured to enhance or modulate diffusion, localization, targeting, active or passive transport, or uptake, for example, of one or more active agents, such as the aminoglycoside. Other pharmaceutically acceptable excipients or additives may include, for example, solvents, preservatives, flavorings, stabilizers (including antioxidants), colorants, lubricant, sorbent, glident, filler, bulking agent, and other additives used in preparations administered into the oral cavity, intranasally, the ear, or via another body orifice. Certain of the formulations wherein the active agents are provided as a dry powder may also include one or more of the pharmaceutically acceptable excipients provided in a dry form such as a powder.

It is to be appreciated that the compositions described herein which employ a base for use in pharmaceutical compounding and manufacturing of topical preparations, may be used for the preparation of compositions for topical administration for appropriate conditions, for example, in some embodiments, the pharmaceutically acceptable excipients or additives include a base for use in pharmaceutical compounding and manufacturing of topical preparations. Such base compositions are known to those skilled in the art. In a preferred embodiment, the base is the PCCA base having the name LoxaSperse. LoxaSperse is an excipient base powder manufactured by PCCA (Houston, Tex.) used as a chemical dispersing or solubilizing agent in irrigation or nebulization formulations, improving the solubility and dispersibility of poorly water soluble Active Pharmaceutical Ingredients (APIs) or agents. LoxaSperse is a blend of specially micronized xylitol with an optimized ratio of micronized poloxamers, designed to improve the dispersibility and solubility of APIs.

In various embodiments, the dry powder formulation comprises an aminoglycoside such as tobramycin and a mucolytic and one or more pharmaceutically acceptable excipients. In one embodiment, the dry powder formulation includes or may be combined with excipients comprising a humectant, emollient, dispersing, suspending, or solubilizing agent configured to increase the dispersibility or solubility of the aminoglycoside or mucolytic in a base solution. As described above, in a preferred embodiment, the aminoglycoside comprises tobramycin and the mucolytic comprises acetylcysteine. In this or another embodiment, the composition may comprise the pharmaceutically acceptable excipient base powder LoxaSperse. The composition may be provided in a unit dose or dosage form comprising the dry powder formulation in a compressed tablet form, as a loose powder, or encapsulated within a capsule. In various embodiments, the aminoglycoside, mucolytic, dry excipients such as LoxaSperse, for example, may be formulated for subsequent addition to a base such as an aqueous medium to form an aqueous formulation or dosage form.

The unit dose or dosage form may be configured for one or more routes of administration. For example, in one embodiment, the unit dose or dosage form is configured as a suppository, such as a rectal, urethral, or vaginal suppository, or may be formed into a topical composition to be applied at a body orifice. The unit dose or dosage form may be formulated for subsequent addition to a base prior to administration to form a dosage form suitable for additional routes of administration. For example, the dry formulation in the form of a tablet, capsule, or powder may be compounded or mixed with a base cream, gel, ointment, oil, or liquid to form an administration solution or dosage form comprising the dry formulation or the components thereof within the base. Those skilled in the art will appreciate that such compounding to produce such solutions may produce administration solutions or dosage forms comprising emulsions, mixtures, or suspensions, for example, that may include the dry formulation wetted, dissolved, solubilized, dispersed, suspended, encapsulated or otherwise associated with or contained within the base.

In various embodiments, a method of treating an infection of the lower or upper respiratory tract comprises prescribing, formulating, or administering to a patient any of the herein described pharmaceutical compositions or formulations prepared therefrom. In various embodiments, a method of treating an upper or lower respiratory tract infection with an aminoglycoside, such as tobramycin, includes delivering the aminoglycoside to the lower respiratory tract wherein the delivering comprises locating the composition at a body orifice and administering the composition thereto, e.g., intranasally.

The composition may be formulated for topical administration at the infection site, e.g., the lungs of the patient. The formulation may be prepared by forming or combining the dry powder formulation comprising one or more of the active agents. In some embodiments, the active agents may be derived from, e.g., obtained from, bulk sources and combined to form the dry powder formulation. The dry powder formulation may be prepared in a dry dosage form suitable for dispensing to a user. The composition may also comprise a unit mixture, dose, or combination thereof prepared from such dry powders, e.g., by addition of the dry powder to a base such as a lotion, oil, cream, ointment, solution. The dry dosage form may be encapsulated, for example, and dispensed for subsequent addition to a base to form a second administration dosage form just prior to administration. Advantages of the dry powder formulation may include increased stability, portability, and customization for a desired administration route or body orifice.

The pharmaceutical composition may include the aminoglycoside or the aminoglycoside and one or more additional active agents in the form of a dry powder formulation. The composition may be dispensed in an encapsulated dry powder format. The composition may comprise the dry powder compressed to form a tablet or may be provided as a loose powder. The composition may further comprise the dry powder encapsulated to provide a dispensable dosage form comprising an encapsulated unit or unit dose. The dry powder may include the aminoglycoside, such as tobramycin, alone or with one or more additional active ingredients or excipients, as described herein, and may be prepared and dispensed in the form of a capsule or tablet. The capsule may be any suitable capsule. In one embodiment, the capsule may be a hard gelatin capsule filled with the dry powder of the formulation by introducing the material into one section of the capsule and capping it with a second section. Other forms of encapsulation may include coating or wrapping the dry powders in unit dose quantities, e.g., within a film, coating, or other containment unit, which may or may not be suitable for ingestion or consumption. The capsule contents may be removed for reconstitution into a liquid or viscous form for topical administration of the composition. Alternatively, the capsule may be of a type that dissolves in a reconstitution liquid to form a solution, suspension, cream, gel, or ointment. The capsule may also be configured to dissolve, breakdown, or otherwise release the dry formulation at the one or more body orifices upon location at or into the body orifice, e.g., via action of or interaction with body fluids or enzymes present at the orifice.

Prior to administration the composition may be crushed into a powder, when in tablet form, or opened to release the dry powder, when in capsule form. In a further embodiment, a user may mix the dry formulation, e.g., the contents of a capsule or crushed tablet, with a desired base or diluent liquid configured for topical administration, e.g., sterile water, water for injection, sodium chloride solution, topical cream, gel, or ointment, or mouth wash.

In various embodiments, the pharmaceutical composition in a unit dose or dosage form may comprise a suppository for insertion into a body orifice, such as the rectum, vagina, or urethra. In some embodiments, the composition may comprise a dosage form including a capsule, tablet, gum, lozenge, or pouch configured for sublingual or sublabial administration. The body cavities or orifices may comprise one or more of the nasal cavity, nostrils, tear duct, anus, vagina, urethra, mouth, and ear canal. In various embodiments, the dry powder may be removed from the capsule prior to administration and further formulated into a topical formulation comprising a solution, suspension, mixture, cream, oil, gel, ointment, lozenge, or gum as also described herein. Such dosage forms may be delivered, for example, by application, dissolution, or irrigation at a body cavity or orifice or topically to skin or a mucosal surface, e.g., drops applied to tear ducts or a mouth rinse, wash, or gargle composition.

In various embodiments, a method of treating an upper or lower respiratory tract infection includes administering a pharmaceutical composition comprising an aminoglycoside or an aminoglycoside and one or more additional active agents or pharmaceutically acceptable excipients or additives. As described above, in some embodiments, the aminoglycoside comprises tobramycin, the one or more additional active agent comprises a mucolytic agent such as acetylcysteine, and the one or more pharmaceutically acceptable excipients or additives may include LoxaSperse. Administering the composition may comprise locating the pharmaceutical composition at a body orifice for topical or localized administration directly to the infected area or areas.

According to one method, the pharmaceutical composition is formulated for administration by mixing the dry powder with an aqueous base such as water for injection or a suitable sodium chloride solution, e.g., a 0.9% sodium chloride solution, just prior to administration, which may include the dry powder mixed, dissolved, suspended, or dispersed within the base. Administration of the formulation may be by inhalation of the dry powder or by nebulization of a dosage form comprising the nebulizer solution formed from the dry powder added to the aqueous base.

In one embodiment, a method of preparing the pharmaceutical composition for delivery to a target site to treat an upper or lower respiratory tract infection is disclosed. The target site may be an infected tissue or surface thereof. According to various embodiments, the treatment of an upper or lower respiratory tract infection may comprise delivery of the pharmaceutical composition to the upper or lower respiratory tract for topical or local action at the target site, e.g., infected lungs, via nebulization of a solution comprising the pharmaceutical composition wherein the composition comprises a pharmaceutically effective dose of the aminoglycoside tobramycin and one or more additional active agents such as the mucolytic acetylcysteine. For example, tobramycin may be included in an amount approximately 100 mg per unit, e.g., capsule, or approximately 150 mg tobramycin sulfate per unit and acetylcysteine may be included in an amount approximately 20 mg per unit. However, other combinations may be used. For example, tobramycin may be included in an amount by weight greater than acetylcysteine. Tobramycin may be included in a ratio by weight greater than 1:1, 2:1. 3:1, 4:1, 5:1, 6:1, or anywhere therebetween with respect to acetylcysteine. The formulation may also comprise one or more pharmaceutically acceptable excipients, which may include LoxaSperse. The composition may be formulated and prepared for topical administration according to the methodologies described herein.

The nebulization solution dosage form may comprise the unit dose quantity of active ingredients within a suitable volume of base. In various embodiments, the suitable volume is that which is suitable for administration of the composition via nebulization. In one embodiment, the suitable volume is that which is suitable for administration via in a small volume nebulizer, which may be an intranasal nebulizer. In another embodiment, a suitable volume is that which is suitable for nasal irrigation.

As introduced above, the pharmaceutical composition may be mixed with a suitable base to produce an intranasal dosage form. In one embodiment, the intranasal dosage form comprises a nebulizer solution dosage form configured for nebulization and intranasal administration to deliver the composition active agents and components to the upper or lower respiratory tract. Such a dosage form may include aqueous solutions for nebulization and delivery by an intranasal nebulizer device. In some arrangements, an advantage of intranasal nebulizer delivery includes simple tidal breathing may be sufficient to receive the designed dose of the pharmaceutical, which may increase patient compliance. Intranasal administration via nebulization of the formulation may efficiently deliver the nebulized composition to the respiratory tract, e.g., the nasal and paranasal sinus cavities. Once delivered, deposits of the formulation or its active agents, which may be in a concentrated state, may form on surfaces of the respiratory tract. Delivery of the active agents using a nebulizer may enhance delivery efficiency and treatment options by providing flexibility with respect to personalization or customization of treatment. Nebulization may be by any suitable commercially available nebulizer device, preferably an intranasal nebulizer. Nebulization delivery systems may beneficially avoid side effects associated with cold medicated irrigations or aerosol-generating powered nasal irrigators.

In one embodiment, the pharmaceutical composition may be mixed with the suitable base to produce a nebulizer solution dosage form for small particle nebulization using a small particle nebulization delivery system. For example, the nebulizer solution dosage form may be formulated for inhalational delivery to the lungs via a small particle size nebulizer, such as a PARI or Sinustar™, Omron, or other nebulizer configured to nebulize the dosage form to produce particles or droplets less than 10 microns in size thereby allowing penetration into the lower respiratory tract, e.g., the lungs. In one embodiment, the small particle nebulization delivery system may be configured to nebulize the formulation to produce small particles or droplets, e.g., having aerosol characteristics, wherein the majority of the particles or droplets formed by the nebulization are less that 5 microns. In some embodiments, 60%, 70%, 80%, or greater of the particles or droplets are less than 5 microns. In various embodiments, nebulization with a small particle nebulizer produces nebulized aerosol particles wherein the majority of particles are less than 10 microns, 8 microns, 5 microns, or 3 microns. In these or other embodiments, the nebulized particles may be produced within a particle size dispersion wherein at least 50%, 60%, 70%, 80%, 90%, or 95% of the particles may be within about 3 microns and about 10 microns, about 3 microns and about 8 microns, about 3 microns and about 5 microns, about 5 microns and about 8 microns, about 5 microns and about 10 microns, or about 8 microns and about 10 microns. Accordingly, a method of administering a nebulizer formulation or dosage form may comprise using a small particle nebulization delivery system and nebulizing the formulation to form small particles or droplets. The nebulized small particles of the formulation may then be inhaled into the upper airway and deposit thereafter at the paranasal sinus and nasal mucosa.

Compared to large particle nebulization delivery systems, small particle nebulization delivery systems deliver a greater fraction of drug to the pulmonary system. This may increase systemic bioavailability of the active agents or drugs. Thus when increased systemic bioavailability is not desirable, e.g., when such bioavailability is linked to unwanted side effects, the formulation may be prepared for and delivered by a large particle nebulization delivery system. In at least one embodiment, treatment of an upper or lower respiratory tract infection comprises nebulizing the nebulizer formulation to produce both small and large particles to target desired respiratory surfaces.

In various embodiments, the pharmaceutical composition comprises the aminoglycoside, such as tobramycin, and one or more additional active agents. The one or more additional active agents may comprise one or more of a surface-active-agent, such as colistimethate, or a mucolytic agent, such as acetylcysteine, a quinolone, such as a third generation quinolone, e.g., levofloxacin. In one such embodiment, a combinational dry powder formulation comprises tobramycin, colistimethate, and levofloxacin. Tobramycin may be present in a unit dose at between about 50 to about 300 mg, such as about 50 mg, 75 mg, 100 mg, 125 mg, 150 mg, 175 mg, 200 mg, 225 mg, 250 mg, 275 mg, or 300 mg. Levofloxacin may be present in the unit dose at between 50 to 300 mg, such as about 50 mg, 75 mg, 100 mg, 125 mg, 150 mg, 175 mg, 200 mg, 225 mg, 250 mg, 275 mg, or 300 mg. In some embodiments, Tobramycin is present in an amount the same or greater than levofloxacin. For example, in one embodiment, tobramycin is present in an amount of between about 80 mg and about 125 mg and levofloxacin is present in an amount about the same or less than that of tobramycin. According to one method, the above combinational dry powder formulation is configured to be mixed with a base liquid to form a nebulizer solution dosage form for the treatment of a respiratory tract infection such as a lower respiratory tract infection caused by the organism Pseudomonas aeruginosa. In one such embodiment, a method of treatment of a lower respiratory tract infection comprises administering the nebulizer solution dosage form prepared from the dry powder using a small particle nebulizer for targeted delivery and deposition of the inhaled small particles along the lower respiratory tract. For example, the treatment may comprise intranasal inhalation of the small particles generated using the small particle nebulizer, which may be a intranasal nebulizer.

In one embodiment, the dry powder dosage form may be mixed with the suitable base liquid to form a nebulizer solution dosage form for large particle nebulization using a large particle nebulization delivery system. In some instances, a nebulizer solution dosage form may be suitable for both large and small particle nebulization. In various embodiments, the large particle nebulizer may be configured to deliver the nebulized solution intranasally. The large particle nebulization delivery system may produce particles or droplets of the solution wherein a majority of such particles or droplets are larger than about 5 microns, about 10 microns, about 15 microns, about 20 microns or more, such as about 23 microns. In these or other embodiments, the nebulized particles may be produced within a particle size dispersion wherein at least 50%, 60%, 70%, 80%, 90%, or 95% of the particles may be within about 10 microns and about 25 microns, about 10 microns and about 20 microns, about 10 microns and about 15 microns, about 15 microns and about 25 microns, about 15 microns and about 20 microns, or about 20 microns and about 25 microns. Accordingly, a method of administering such a formulation may comprise using a large particle nebulization system and nebulizing the formulation to form large particles. The nebulized large particles of the formulation may then be inhaled intranasally into the nasal and paranasal sinus cavities for deposition on the frontal recess/sinus, spheno-ethmoid recess, ethmoid cavity, sphenoid and maxillary sinuses, turbinates, middle meatus, and olfactory cleft. The large particles nebulization system may provide low volume, high concentration delivery of the formulation.

While any suitable large particle nebulization system or device may typically be used to nebulize the formulation for administration to the patient, one suitable device is a NasoNeb® Nasal Nebulizer. The large particle delivery system may be employed to administer the formulation to the patient to deliver a deep, penetrating aerosol to the nasal and paranasal sinus cavities of the patient. Such delivery may include little to no incidental pulmonary delivery of drugs, which may otherwise occur in small particle systems as described above. For example, in some embodiments, large particle nebulization may provide superior outcomes compared to small particle nebulization, which typically include pulmonary delivery and decreased nasal and paranasal sinus cavity disposition.

In one embodiment, the large particle nebulization system is configured to nebulize the formulation to generate large particles comprising the formulation for delivery of the large particles via a positive pressure airstream that ensures the formulation components reach all of the desired nasal and paranasal sinus cavities. The large particle nebulization system also preferably delivers the large particles such that they are readily filtered by the nose to ensure a large percentage of medication is delivered where intended and that little or no unintended formulation components are delivered to the lungs, reducing the risk of unwanted complications.

In one embodiment, the large particle nebulization system is configured to deliver the composition in a low volume base to ensure that the medication of the formulation stays in the nasal cavity or is deposited on respiratory surfaces in high concentration droplets. In one embodiment, the large particle nebulization system is configured to deliver between about 0.2 mL to about 15 mL of the nebulizer dosage form for retention in the nasal and paranasal sinus cavities. In one embodiment, the large particle nebulization system may also reduce complications associated with repeated exposure to cold fluid irrigation such as exostoses of the paranasal sinus cavities by warming the formulation to near room temperature upon nebulization, which may help to avoid the iatrogenic complication of exostoses from cold fluid irrigation.

Administering the nebulizer formulation via nebulization in the form of large particles delivered to the upper respiratory tract by large particle nebulizer devices or delivery systems may avoid undesirable complications that may be linked to long term use of small particle nebulization systems such as vocal irritation/alterations, chronic cough, antimicrobial resistance, eosinophilic pneumonia, and reduced lung function.

According to one embodiment, the encapsulated dry powder dosage form may be formulated as described above and dispensed to a user, such as a medical professional, patient, or assistant. For example, in one embodiment, one or more encapsulated unit doses in a dry powder format may be dispensed to the user. The user may be instructed to prepare the composition for administration just prior to administration. For example, the user may be instructed to combine the unit dose with a base such as a commercially available solution of a sterile liquid, water for injection, or 0.9% sodium chloride solution for administration of the composition via a nebulizer. The nebulizer may be configured for delivery of the composition to the lungs. The nebulizer may be a large or small particle size nebulizer, which, according to some embodiments, may include an intranasal large or small particle nebulizer.

In some embodiments, a method of treating an upper or lower respiratory tract infection comprises therapeutic action at the site of application or along the administration pathway to the target site, e.g., at or near the lungs, oral cavity, pharynx, trachea, skin, or body orifice, in addition to therapeutic action at the delivery site. Other active agents, for example, may be included in the pharmaceutical composition or added thereto, e.g., within a base, during preparation or just prior to administration for purposes of alleviating other undesirable conditions along the administration pathway, which may or may not be associated with the respiratory infection. Such agents may include, for example, anesthetics, analgesics, anti-inflammatories, antibacterial agents, antiviral agents and emollients.

Administration may include respiratory routes to deliver the active agents to the target site such as by inhalation or nebulization, as described above. In some embodiments, the composition may be configured for administration at a body orifice and may include intranasal, mucosal, ophthalmic, oral buccal, oral sublingual, rectal, cutaneous, intravaginal, or intravesical administration. In one embodiment, a method of administering the composition may include inserting the dry formulation at a body orifice such that the formulation may be delivered therein, e.g., upon disintegration of a tablet or dissolution of a capsule containing the dry powder formulation. Administering the composition may comprise locating the composition at a body orifice for topical localized administration directly to the infected area or areas. In certain embodiments, the dry formulation may be formulated for topical administration including one or more of insufflation, sublingual, sublabial, or vaginal. For example the composition may be configured for administration at a body orifice comprising one or more of a nasal cavity, nostril, tear duct, anus, vagina, urethra, mouth, and ear canal. According to one method, a patient or medical professional may apply or insert the composition at a body orifice. It will be appreciated that in some embodiments the composition is suitable for administration at multiple body orifices. The pharmaceutical composition configured for administration at a body orifice comprising one or more of a nasal cavity, nostril, tear duct, anus, vagina, urethra, mouth, and ear canal may be used to treat local infections at the orifices or in some instances may be used to treat infections at other locations including underlying tissues, semi-local tissues, or even including systemic transport or effects.

In one embodiment, the pharmaceutical composition is prepared for administration to the mouth of a patient. The pharmaceutical composition may comprise an aminoglycoside or aminoglycoside and one or more additional active agents, either of which may further include one or more excipients as described here. For example, the pharmaceutical composition may comprise tobramycin alone or with acetylcysteine or one or more pharmaceutically acceptable excipients such as LoxaSperse or a combination thereof. The composition may include the dry powder ingredients or components thereof formulated for administration to the mouth. In some formulations, the pharmaceutical composition may comprise formulations prepared for sublingual or sublabial administration including pouches, gums, lozenges, capsules, or tablets configured to dissolve or disintegrate upon locating within the mouth and contacting saliva or fluids. In a further example, the compositions described herein may include or be prepared or formulated for administration to a body orifice by incorporation into a mouthwash formulation for administration to the oral cavity. For example, the dry powder comprising the one or more active agents or one or more active agents and excipients, as described above, may be combined with a mouthwash base material. The oral rinse or solution base material may comprise a diphenhydramine elixir such as Benadryl®, or a generic equivalent. In some embodiments, the concentration of diphenhydramine in the elixir is 12.5 mg/5 mL. The base material may also comprise aluminum hydroxide and/or magnesium hydroxide, including Maalox® (MgOH & AlOH; 40 mg/ml), or a generic equivalent, Mylanta, or a generic equivalent. Other agents that can be included in the base material include a local anesthetic such as lidocaine or Dyclone, nystatin, sucralfate, Kaopectate, allopurinol, vitamin E, beta-carotene, Kamillosan liquid, aspirin, antiprostaglandins, prostaglandins, MGI 209 (marketed as Oratect Gel), silver nitrate, and antiviral agents such as acyclovir. Other suitable oral rinse base material may include Prevident® or Phos-Flur® rinses.

In the case of an oral rinse, the user is instructed to mix the contents of 1 capsule or vial with a base material, such as about 10 mL of the desired base material. The preparation is to be swished around in the mouth of the patient for an appropriate length of time, e.g., at least about 1-3 minutes, then expectorated. This is also known a swish/spit methodology.

In the case of an oral solution, the user is instructed to mix the contents of 1 unit dose, such as a capsule or vial, with about 10 mL of the desired base material. The preparation is to be swished around in the mouth of the patient for an appropriate length of time, e.g., at least about 1-3 minutes, then swallowed. Typically, the administration is three times daily.

Also included is a method delivering an aminoglycoside to the lower respiratory tract or lungs of a mammal comprising administering an intranasal formulation. The intranasal formulation may comprise the pharmaceutical composition comprising an aminoglycoside or aminoglycoside and one or more additional active agents, either of which may further include one or more excipients as described here. For example, the pharmaceutical composition may comprise tobramycin alone or with acetylcysteine or one or more pharmaceutically acceptable excipients such as LoxaSperse or a combination thereof. The pharmaceutical composition may be prepared as a dry powder formulation as described herein for subsequent administration, which may include mixing the pharmaceutical composition with a suitable base prior to administration. In various embodiments, the administering of the intranasal formulation may be via insertion of the pharmaceutical composition formulated as a spray, nasal irrigation, topical cream, ointment, gel, or the like, or by insufflation.

Compositions and methods for preparing or administering an intranasal administrable formulation of the pharmaceutical composition may include mixing the dry powder ingredients into a nasal solution or suspension with a diluent suitable for intranasal delivery of active ingredients. For example, a liquid spray, solution, or suspension for irrigation or application to the mucosal surface within the orifice. Diluents for the preparation of intranasal formulations are known to those skilled in the art, for example 0.9% sodium chloride solution, which is available in vials and with a prescription or over the counter.

In other embodiments, the typical mode of administration is in flush form or liquid stream form. An example of suitable sinus rinse delivery mechanisms include the NeilMed® Sinus Rinse Bottle, a medical syringe of about 20 to 60 mL in size, and other squeeze bottle irrigation devices. Typically, the intranasal formulation is administered two or three times a day.

Also included are pharmaceutical compositions for aural or otic administration and methods of preparing the same according to any of the embodiments described herein. For example, the pharmaceutical composition comprising the aminoglycoside or aminoglycoside and the one or more additional active agents, either of which may further include one or more excipients as described herein, in the form of a dry powder may be mixed or combined into a base solution or suspension with a diluent suitable for otic delivery of active agent. Diluents for the preparation of formulations for aural administration are known to those skilled in the art and are similar to those described above for the preparation of intranasal formulations. According to one method, the resulting solution or suspension may be administered to the ear or ear canal. For example, the solution or suspension may be drawn into a syringe and then dispensed into the ear canal. The patient should lie with the ear positioned upward, and then the composition should be instilled. The ear canal should be completely filled to ensure that medication is allowed to touch all areas of the ear canal.

A method of compounding may include combining the active ingredients with a base composition and shaking or otherwise mixing the combined ingredients, sifting the resulting powder mixture through a fine mesh strainer, mixing the powdered mixture until a homogeneous powder results. A suitable mixer for this purpose is a TURBULA® mixer, which is able to mix powdery substances with differing specific weights and particle sizes. The mixing is generally performed for about one hour. The resulting homogeneous mixture can be encapsulated into a suitable capsule, for example a #00 size capsule.

All numerical quantities stated herein are approximate unless stated otherwise, meaning that the term “about” or “approximately” may be inferred when not expressly stated. The numerical quantities disclosed herein may be nominal numerical quantities and are to be understood as not being strictly limited to the exact numerical values recited. Instead, unless stated otherwise, each numerical value is intended to mean both the recited value and a functionally, for example pharmaceutically, equivalent range surrounding that value. All numerical ranges stated herein include all sub-ranges subsumed therein. For example, a range of approximately or about 1 to 10 is intended to include all sub-ranges between and including the recited minimum value of 1 and the recited maximum value of 10. Any maximum numerical limitation recited herein is intended to include all lower numerical limitations. Any minimum numerical limitation recited herein is intended to include all higher numerical limitations. Additionally, in some illustrative embodiments, quantities or ranges may be given. It is to be understood that any such quantity or range is provided as an illustrative example or instance of an embodiment and is not intended to limit that or other embodiments.

This disclosure describes various elements, features, aspects, and advantages of various embodiments of the stopping systems, apparatuses, and methods thereof. It is to be understood that certain descriptions of the various embodiments have been simplified to illustrate only those elements, features and aspects that are relevant to a more clear understanding of the disclosed embodiments, while eliminating, for purposes of brevity or clarity, other elements, features and aspects. Any references to “various embodiments,” “certain embodiments,” “some embodiments,” “one embodiment,” or “an embodiment” generally means that a particular element, feature and/or aspect described in the embodiment is included in at least one embodiment. The phrases “in various embodiments,” “in certain embodiments,” “in some embodiments,” “in one embodiment,” or “in an embodiment” may not refer to the same embodiment.” Furthermore, the phrases “in one such embodiment” or “in certain such embodiments,” while generally referring to and elaborating upon a preceding embodiment, is not intended to suggest that the elements, features, and aspects of the embodiment introduced by the phrase are limited to the preceding embodiment; rather, the phrase is provided to assist the reader in understanding the various elements, features, and aspects disclosed herein and it is to be understood that those having ordinary skill in the art will recognize that such elements, features, and aspects presented in the introduced embodiment may be applied in combination with other various combinations and sub-combinations of the elements, features, and aspects presented in the disclosed embodiments. The present disclosure is not intended to be limited by the percent composition of the examples unless claimed otherwise. Percent compositions are to be understood as being by weight unless specified otherwise.

The phrase “consisting essentially of” limits a composition to the specified materials or steps and those additional, undefined components that do not materially affect the basic and novel characteristic(s) of the composition, such as, for example, additional active ingredients. In still other embodiments, the formulations consist of the listed active ingredients and a pharmaceutically acceptable carrier. “Consisting of” refers to the inclusion of exactly one element of a number or list of elements. The grammatical articles “one”, “a”, “an”, and “the”, as used in this specification, are intended to include “at least one” or “one or more”, unless otherwise indicated. Thus, the articles are used in this specification to refer to one or more than one (i.e., to “at least one”) of the grammatical objects of the article. By way of example, “a component” means one or more components, and thus, possibly, more than one component is contemplated and may be employed or used in an implementation of the described embodiments. Further, the use of a singular noun includes the plural, and the use of a plural noun includes the singular, unless the context of the usage requires otherwise. Additionally, the grammatical conjunctions “and” and “or” are used herein according to their accepted usage. By way of example, “x and y” refers to “x” and “y”. On the other hand, “x or y” refers to “x”, “y”, or both “x” and “y”, whereas “either x or y” refers to exclusivity.

1. The method of claim 19, further comprising encapsulating the dry powder formulation in a capsule.

2. The method of claim 1, wherein the dry powder formulation comprises acetylcysteine and wherein preparing the dry powder formulation comprises combining tobramycin or a pharmaceutically acceptable salt thereof, the excipient base, and acetylcysteine or a pharmaceutically acceptable salt thereof.

3. (canceled)

4. The method of claim 1, wherein encapsulating the dry powder formulation generates a capsule comprising approximately 100 mg tobramycin or approximately 150 mg tobramycin sulfate.

5. The method of claim 1, wherein encapsulating the dry powder formulation generates a capsule comprising approximately 100 mg tobramycin and approximately 20 mg acetylcysteine.

6. The method of claim 1, wherein preparing the dry powder formulation comprises obtaining tobramycin from a bulk source.

7-18. (canceled)

19. A method of treating a patient for an upper or lower respiratory tract infection, the method comprising:
preparing a dry powder formulation for nebulization, wherein the dry powder formulation comprises (i) tobramycin or a pharmaceutically acceptable salt thereof, (ii) an excipient base, and (iii) either acetylcysteine or a pharmaceutically acceptable salt thereof, or levofloxacin or a pharmaceutically acceptable salt thereof;
adding the dry powder formulation to a sodium chloride solution to produce a solution dosage form;
nebulizing the solution dosage form to form particles wherein a majority of the particles are greater than 10 microns; and
intranasally administering to a patient the nebulized solution dosage form to treat an infection of the upper or lower respiratory tract.

20. (canceled)

21. The method of claim 33, further comprising encapsulating the dry powder formulation in a capsule.

22. A method of treating a patient for an upper or lower respiratory tract infection, the method comprising:
mixing a dry powder formulation with a sodium chloride solution to prepare a solution dosage form, wherein the dry powder formulation comprises (i) tobramycin or a pharmaceutically acceptable salt thereof, (ii) an excipient base, and (iii) either acetylcysteine or a pharmaceutically acceptable salt thereof, or levofloxacin or a pharmaceutically acceptable salt thereof;
nebulizing the solution dosage form to form particles wherein a majority of the particles are greater than 10 microns; and
intranasally administering to a patient the nebulized solution dosage form.

23. (canceled)

24. The method of claim 22, wherein the solution dosage form comprises approximately 100 mg tobramycin or approximately 150 mg tobramycin sulfate and approximately 20 mg acetylcysteine.

25. (canceled)

26. The method of claim 1, wherein the dry powder formulation comprises levofloxacin and wherein preparing the dry powder formulation comprises combining tobramycin or a pharmaceutically acceptable salt thereof, the excipient base, and levofloxacin or a pharmaceutically acceptable salt thereof.

27. The method of claim 1, wherein the excipient base is in powder form and comprises micronized xylitol and micronized poloxamers.

28. The method of claim 1, wherein preparing a dry powder formulation comprises obtaining acetylcysteine or levofloxacin from a bulk source.

29-31. (canceled)

32. The method of claim 1, further comprising removing the dry powder formulation from the capsule prior to mixing the dry powder formulation with the sodium chloride solution.

33. The method of claim 19, wherein the excipient base is in powder form and comprises micronized xylitol and micronized poloxamers.

34. The method of claim 22, wherein the dry powder formulation is encapsulated and the method comprises removing the dry powder formulation from a capsule prior to mixing the dry powder formulation with the sodium chloride solution.

35. The method of claim 22, wherein the excipient base is in powder form comprising micronized xylitol and micronized poloxamers.

36. The method of claim 22, wherein the solution dosage form comprises approximately 160 mg tobramycin and approximately 125 mg levofloxacin.

37. The method of claim 19, wherein at least 80% of the particles are between 10 microns and 25 microns.

38. The method of claim 19, wherein at least 80% of the particles are between 15 microns and 25 microns.

39. The method of claim 19, wherein a majority of the particles are greater than 20 microns.

40. The method of claim 22, wherein at least 80% of the particles are between 10 microns and 25 microns.

41. The method of claim 22, wherein at least 80% of the particles are between 15 microns and 25 microns.

42. The method of claim 22, wherein a majority of the particles are greater than 20 microns.
